SEQUENCE LISTING (25 sequences)

SEQ ID NO: 1 - protein, 29 aa, Artificial Sequence
  rat nNOS
NSVSCSDPLVSSWRRKRKESSNTDSAGAL

SEQ ID NO: 2 - protein, 29 aa, Artificial Sequence
  bovine eNOS
NSVSCSDPLVSSWRRKRKESSNTDSAGAL

SEQ ID NO: 3 - protein, 29 aa, Artificial Sequence
  Human MK2-B
IKKIEDASNPLLLKRRKKARALEAAALAH

SEQ ID NO: 4 - protein, 29 aa, Artificial Sequence
  Human MK3
IKDLKTSNNRLLNKRRKKQAGSSSASQGC

SEQ ID NO: 5 - protein, 20 aa, Artificial Sequence
  Human MKK6
MSQSKGKKRNPGLKIPKEAF

SEQ ID NO: 6 - DNA, 166 bp, Artificial Sequence
  Human nNOS
tgaggcaccccaactctgtgcaggaagaaaggaagagctacaaggtccgattcaacagcgtctcctcctactctgactcccaaaaatcatcaggcgatgggcccgacctcagagacaactttgagagtgctggacccctggccaatgtgaggttctcagtttttgg

SEQ ID NO: 7 - DNA, 166 bp, Artificial Sequence
  rat nNOS
tgaggcaccccaactctgtgcaggaggagagaaagagctacaaggtccgattcaacagcgtctcctcctattctgactcccgaaagtcatcgggcgacggacccgacctcagagacaactttgaaagtactggacccctggccaatgtgaggttctcagtgttcgg

SEQ ID NO: 8 - DNA, 175 bp, Artificial Sequence
  Human eNOS
tgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgg

SEQ ID NO: 9 - DNA, 175 bp, Artificial Sequence
  bovine eNOS
tgtcggggccctacaacagctccccgcggccggaacagcacaagagttacaagatccgcttcaacagcgtctcctgctcagacccgctggtgtcctcctggcggcggaagagaaaggagtccagcaacacagacagtgcgggggccctggggaccctcaggttctgtgtgttcgg

SEQ ID NO: 10 - DNA, 40 bp, Artificial Sequence
  Human iNOS
tgaaagagctcaacaacaaattcaggtacgctgtgtttgg

SEQ ID NO: 11 - protein, 55 aa, Artificial Sequence
  Human nNOS
RHPNSVQEERKSYKVRFNSVSSYSDSQKSSGDGPDLRDNFESAGPLANVRFSVFG

SEQ ID NO: 12 - protein, 55 aa, Artificial Sequence
  rat nNOS
RHPNSVQEERKSYSVRFNSVSSYSDSRKSSGDGPDLRDNFESTGPLANVRFSVFG

SEQ ID NO: 13 - protein, 58 aa, Artificial Sequence
  Human eNOS
SGPYNSSPRPEQHKSYKIRFNSISCSDPLVSSWRRKRKESSNTDSAGALGTLRFCVFG

SEQ ID NO: 14 - protein, 58 aa, Artificial Sequence
  Bovine eNOS
SGPYNSSPRPEQHKSYKIRFNSVSCSDPLVSSWRRKRKESSNTDSAGALGTLRFCVFG

SEQ ID NO: 15 - protein, 13 aa, Artificial Sequence
  Human iNOS
KELNNKFRYAVFG

SEQ ID NO: 16 - protein, 31 aa, Artificial Sequence
  eNOS tryptic digests
RAPAPATPHAPDHSPAPNSPTLTRPPEGPKF

SEQ ID NO: 17 - protein, 31 aa, Artificial Sequence; identical to SEQ ID NO: 16
  eNOS tryptic digests

SEQ ID NO: 18 - protein, 31 aa, Artificial Sequence; identical to SEQ ID NO: 16
  eNOS tryptic digests

SEQ ID NO: 19 - protein, 34 aa, Artificial Sequence
  eNOS tryptic digests
RAPAPATPHAPDHSPAPNSPTLTRPPEGPKFPRV

SEQ ID NO: 20 - protein, 31 aa, Artificial Sequence; identical to SEQ ID NO: 16
  eNOS tryptic digests

SEQ ID NO: 21 - protein, 23 aa, Artificial Sequence
  eNOS tryptic digests
FAAALMEMSGPYNSSPRPEQHKS

SEQ ID NO: 22 - protein, 21 aa, Artificial Sequence
  eNOS tryptic digests
AALMEMSGPYNSSPRPEQHKS

SEQ ID NO: 23 - protein, 21 aa, Artificial Sequence
  eNOS tryptic digests
AALMEMSGPYNSSPRPEQHKS

SEQ ID NO: 24 - protein, 19 aa, Artificial Sequence
  eNOS tryptic digests
LMEMSGPYNSSPRPEQHKS

SEQ ID NO: 25 - protein, 26 aa, Artificial Sequence
  eNOS tryptic digests
ATPHAPDHSPAPNSPTLTRPPEGPKF

(19) United States
(12) Patent Application Publication
(10) Pub. No.: US 2013/0224827 A1
(43) Pub. Date: Aug. 29, 2013

(54) ENDOTHELIAL NITRIC OXIDE SYNTHASE

(71) Applicants: John C. Salerno, Acworth, GA (US); Carol A. Chrestensen, Marietta, GA (US)

(72) Inventors: John C. Salerno, Acworth, GA (US); Carol A. Chrestensen, Marietta, GA (US)

(21) Appl. No.: 13/773,721

(22) Filed: Feb. 22, 2013

Related U.S. Application Data
(60) Provisional application No. 61/637,124, filed on Apr. 23, 2012.
(60) Provisional application No. 61/602,450, filed on Feb. 23, 2012.

(51) Int. Cl. C12N 9/99; C07K 16/40; C12N 9/96; C12N 9/00

(52) U.S. Cl. CPC C12N 9/99; C12N 9/0075; C07K 16/40; C12N 9/96
USPC 435/184; 435/189; 530/300; 530/387.9; 435/188; 435/375

(57) ABSTRACT

The activity of endothelial nitric oxide synthase (eNOS) was modulated by contact with an effective amount of a mitogen-activated protein (MAP) kinase, resulting in phosphorylation of S602, T46, and/or S58. Kinetics and stoichiometry are disclosed. The contact strongly reduced nitric oxide (NO) synthesis, and also inhibited the cytochrome c reductase activity of eNOS reductase domains. Three sites of phosphorylation were determined that matched the serine-proline (SP) and threonine-proline (TP) motifs of typical MAP kinase phosphorylation sites.

### CROSS REFERENCE TO RELATED APPLICATIONS

This application claims priority to U.S. Provisional Application Ser. Nos. 61/602,450 filed Feb. 23, 2012, and 61/637,124 filed Apr. 23, 2012, each of which is expressly incorporated by reference herein in their entirety.

This invention was made with government support under NIH 3R15GM080701-01, and NSF 1020261 and 0950920. The government has certain rights in the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows alignment of the pentabasic recognition site (bold) in endothelial nitric oxide synthase (eNOS) within the D-site for binding partners for extracellular signal-regulated kinase (ERK), p38 MAP kinase, and c-Jun N-terminal kinase (JNK).

FIGS. 2A-B show binding of eNOS to p38.

FIGS. 3A-C show binding of eNOS to ERK-2.

FIG. 4 shows sequence alignment of NOS mammalian isoforms in the autoinhibitory (Al) element region.

FIG. 5 shows the mass spectroscopy results for trypsin digest of ERK phosphorylated eNOS.

FIGS. 6A-B show structural cartoons of NOS reductase domains (FIG. 6A) and eNOS oxygenase dimer (FIG. 6B).

FIGS. 7A-D show an optical biosensing analysis of CaM-eNOS binding.

FIG. 8 shows fluorescence decays of unphosphorylated and ERK phosphorylated eNOS in the presence and absence of calmodulin (CaM).

Endothelial nitric oxide synthase (eNOS) contains a motif similar to recognition sequences in known mitogen activated protein kinase (MAPK) binding partners that is associated with a major eNOS control element. Purified ERK and p38 phosphorylated eNOS with an apparent stoichiometry of 2-3 phosphates per eNOS monomer. Phosphorylation strongly reduced nitric oxide (NO) synthesis and inhibited the cytochrome c reductase activity of the eNOS reductase domains. Three sites of phosphorylation were detected in mass spectroscopy experiments using tryptic digests; all matched the serine-proline (SP) and threonine-proline (TP) motifs of typical MAP kinase phosphorylation sites. As determined by optical biosensing, eNOS bound p38 and ERK with about 100 nM affinity and complex kinetics. Binding was diffusion-limited (kₒₙ about 0.15×10⁶M⁻¹ sec⁻¹). Neuronal NOS also bound p38 but exhibited much slower and weaker binding. p38-eNOS binding was inhibited by calmodulin (CaM). Evidence for a ternary complex was found when eNOS-bound p38 was exposed to CaM, increasing the apparent dissociation rate. These data strongly suggested a direct role for MAPK in regulation of NOS with implications for signaling pathways including angiogenesis and control of vascular tone.

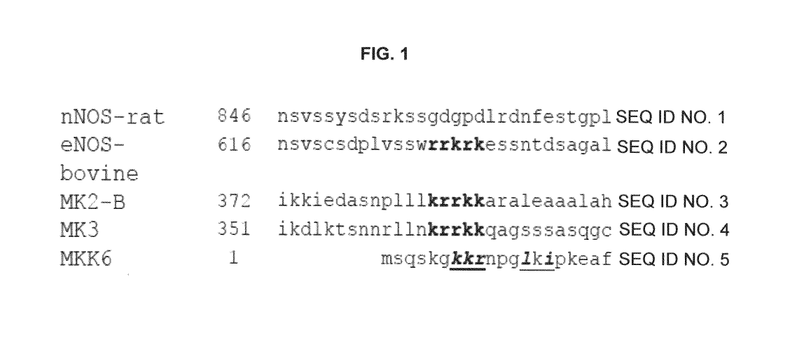

The family of mitogen activated protein kinases (MAPK) are central nodes in stress activated signaling networks regulating gene expression in response to environmental changes, inflammatory cytokines, and other signals. Endothelial nitric oxide synthase (mitogen activated protein kinase 1 and 2 (ERK1/2), p38 and endothelial nitric oxide synthase (eNOS)-generated nitric oxide (NO) are involved in processes such as angiogenesis, ischemia-reperfusion and insulin response (1-10). Experimental evidence indicates that ERK1/2 phosphorylates eNOS in a bradykinin-sensitive ERK1/2-murine thymoma viral oncogene homolog 1 (a.k.a, protein kinase B, AKT)-eNOS-Raf-1 complex in bovine aortic endothelial cells (BAECs) (11). p38 is stimulated by upstream kinases by several pathways and activates a broad spectrum of downstream kinases, including MAPKAP kinases 2 and 3 and numerous transcription factors such as activating transcription factor (ATF) 1/2 and myocyte enhancer factor (MEF)2A (3,12). Many binding partners for ERK, p38 and JNK contain a basic recognition site within the D-site (FIG. 1) that facilitates interaction between MAP kinases and other signaling components (1-4, 12). FIG. 1 shows alignment of the pentabasic sequence (bold) in eNOS and the aligned region in neuronal nitric oxide synthase (nNOS) and with the D sites of MK2-B, MK3, and MKK6. The classic D site sequence is characterized by (K/R2-3-X1-6-φ-X-φ) (4), is bolded and underlined in the MKK6 sequence. MK2-B and MK3 lack the hydrophobic portion of the D site, similar to eNOS. All sequences are human unless otherwise designated; the sequence corresponding to the recognition region is essentially invariant within each group of mammalian kinase and NOS orthologs.

The canonical binding site for recognition by MAP kinases is KKRxxxLxl, but considerable variation from this motif has been postulated to facilitate recognition by a helix and neighboring regions on MAPKs that bear an array of acidic and hydrophobic residues. Among several alternatives, Enslena et al proposed that (R/K)₂-(x)₂₋₆-(L/I)x(L/I) is a more general motif (13). Sharrocks et al presented an even more general scheme for recognition of substrates by MAP kinases involving a multibasic region, an LxL region, and flanking C and N terminal hydrophobic residues; at least two of these four determinants were present in all sequences considered (14). More recently a series of peptides based on putative recognition sites has been used to probe the specificity of a variety of MAP kinases. Variations within the D site contribute to, but do not completely account for, differential binding of related substrates to JNK, ERK and p38 type kinases, all of which recognize these motifs (15).

The endothelial nitric oxide synthase (eNOS) is an important signal generator involved in control of vascular tone, insulin secretion, and angiogenesis (6-10). The primary controller of eNOS is displacement of an autoinhibitory element by calcium/calmodulin (Ca⁺²/CaM) (16), but eNOS is also regulated by several kinases acting at different sites to activate or inhibit NO production (17-19). eNOS regulation also involves targeting to different compartments by myristolation, palmitoylation, and protein-protein interactions. Some of the activating phosphorylation sites are within the autoinhibitory element (S617 and S633 in bovine eNOS), and are phosphorylated by protein kinase A (PKA) and AKT.

The autoinhibitory element contains a conserved region common to CaM regulated NOS enzymes; this includes the phosphorylation sites and a helical region that forms hydrogen bonds with the FMN binding domain and with the two domain dehydrogenase unit with which it is associated in the available crystal structure (20). The C terminal half of the autoinhibitory element is not conserved at the protein level because of local frame shifts which generated a unique pentabasic sequence in mammalian eNOS. As shown in FIG. 1, the corresponding region of nNOS contains only two basic residues, although there are other basic residues, notably a triplet nearby in the canonical calmodulin-binding motif.

Biosensor experiments provide a powerful and versatile probe of protein-protein interactions, allowing verification of predicted binding and quantitative description of affinity and kinetics of binding and release. The inventors utilized optical biosensing to elucidate calcium dependent changes to the rates and affinity of calmodulin binding to NOS (21),

The inventors herein disclose that very compact versions of the D site can be detected and bind with high affinity to MAP kinases such as p38 and ERK1/2. In particular, ERK1/2 binds eNOS but not nNOS and p38 strongly binds to endothelial nitric oxide synthase, but makes a much weaker complex with the neuronal isoform, consistent with recognition of a pentabasic sequence in eNOS by p38. p38-eNOS binding is inhibited and release stimulated by CaM, suggesting a weak ternary complex. Inhibition of eNOS by p38 is consistent with partial competition with CaM.

Signaling networks including elaborate feedback and feedforward mechanisms are responsible for homeostasis over multiple levels of organization. The familiar picture of signal transduction cascades in which amplification is obtained by sequential phosphorylation/activation has been augmented by the discovery of multi-component signaling complexes that may include receptors, kinases, scaffolds and adaptors, and non-kinase signal generators.

Endothelial nitric oxide synthase is a signal generator in the regulation of vascular and airway tone, insulin secretion, angiogenesis and cardiac function, Primary control is through calcium/calmodulin activation, but numerous other inputs have been described including inhibitory and activating phosphorylation by specific kinases, protein-protein interactions, and cell trafficking. Examples of activating kinases include PKA which phosphorylates S1177 and S633, and murine thymoma viral oncogene homolog 1 (a.k.a, protein kinase B) (AKT), which phosphorylates S1177 and S615. S1117 is located in the C terminal tail, which acts to restrict the rate of heme reduction by the flavin containing domains. S633 and S617 are located in a large autoinhibitory insertion in the FMN binding domain, which is displaced by CaM binding during activation. PKC inhibits eNOS by phosphorylating T495 adjacent to the CaM binding site, which interferes with CaM binding mediated activation. Other phosphorylation sites associated with the oxygenase domain are less well studied.

MAP kinases including the ERK, p38 and JNK families are important signaling nodes in pathways that control metabolism, growth and expression. ERK and p38 both function in signaling pathways that involve eNOS, and good evidence for direct phosphorylation of eNOS by ERK1/2 in BAECs has been obtained. Contradictory reports of the site of ERK phosphorylation have appeared.

The present invention discloses a pentabasic binding site for MAP kinases in the eNOS autoinhibitory element as well as targets of phosphorylation, the effects on activity, and the mechanism by which phosphorylation regulates NO synthesis.

The present invention discloses peptides that bind to a pentabasic binding site for MAP kinases in the eNOS autoinhibitory element and which may inhibit the binding of MAP kinases.

Embodiments disclose methods for modulating nitric oxide synthase activity by contacting a nitric oxide synthase with an effective amount of peptide for a time sufficient to allow binding of the nitric oxide synthase with the peptide, where contacting the nitric oxide synthase with the peptide results in binding of the peptide to a pentabasic sequence in the auto-inhibitory element of the nitric oxide synthase. Binding of the peptide to the nitric oxide synthase results in modulation of nitric oxide synthase activity.

Embodiments disclose methods for modulating nitric oxide synthase activity by contacting a nitric oxide synthase with an effective amount of a MAP kinase, resulting in phosphorylation of at least one amino acid selected from the group of S602, T46, and S58. Such amino acid phosphorylation results in modulation of nitric oxide synthase activity.

The present invention discloses methods for modulating the cytochrome C reductase activity of a nitric oxide synthase. A nitric oxide synthase reductase domain is contacted with an effective amount of a MAP kinase, where phosphorylation of at least one amino acid from the nitric oxide synthase reductase domain results in the modulation of cytochrome C reductase activity.

Embodiments of the present invention also disclose pentabasic peptides and variants thereof.

Embodiments of the present invention also disclose antibodies that bind to a pentabasic binding site for MAP kinases in the eNOS autoinhibitory element.

The present invention also discloses antibodies to phosphorylated peptide analogs that may be used to detect phosphorylation of eNOS by ERK or other kinases at the sites that include, but are not limited to, S602 in bovine eNOS (S600 in human eNOS), T46, and S58 in bovine eNOS (T44 and S56 in human eNOS). These antibodies may be raised using phosphorylated (e.g., by ERK) synthetic peptides with sequences derived from the region around the site.

Embodiments of the invention include peptidomimetic mutant enzymes with D or E substituted for the phosphorylated S and T residues that may mimic constitutively phosphorylated enzymes.

Embodiments of the invention include peptidomimetic null enzymes with another residue, commonly A for the phosphorylated S and T residues, which would provide enzymes unphosphorylated at that site.

Embodiments of the invention includes mutated enzymes as described above combined with mimic and null mutants at other sites (e.g. S617 or T497), as well as genes coding for these enzymes.

Embodiments of the invention include vectors and expression systems containing genes encoding the enzymes described herein. Such vectors and expression systems may be incorporated into expression systems for use in cell or tissue culture or in animals or humans. Such vectors may also be used in gene therapy methods (e.g., ERK phosphorylation nulls for higher NO production in vivo).

NOSs were expressed and purified as known in the art (22-24). Heme and flavin content were estimated spectrophotometrically, and activity assayed by following NADPH consumption at 340 nm (23, 24). Assays of eNOS NO synthase activity and cytochrome c reduction were performed as known in the art (25, 26), the concentration of CaM was reduced to 0.6 pM because of potential competition between CaM and p38. Phosphorylated His-p38 was expressed as known in the art (26) and purified using Talon™ resin as directed by the manufacturer. ERK-2 was purchased from PROSPEC (NJ). All biolayer interferometry (27) measurements were made on a ForteBio (Menlo Park Calif.) Octet QK biosensor using streptavidin sensors. Assays were performed in 200 μL volumes at 25° C. p38 and ERK were biotinylated as known in the art (21). Biotinylated kinases were loaded onto sensors for 600 s. After establishing baseline response, kinases were exposed to analyte eNOS or nNOS at a range of concentrations. Baseline, association, and dissociation phases were all performed in NOS buffer (10 mM phosphate, pH 7.5, 100 mM NaCl, 10% glycerol, 0.005% surfactant P-20, 10 uM CaCl₂). Association and dissociation were monitored as nm shift. Nonspecific binding of analytes to sensors without ligand was negligible and so was not subtracted.

Binding and release of analyte was simulated using standard kinetics approaches (21). A single first order model generated acceptable simulations for only the first 30 seconds of binding, but good quality fits were obtained using two components with either sequential (A+B⇄C⇄D) or parallel (A+B⇄C; D+B⇄E) models. Sequential binding was modeled at each analyte concentration with pseudo first order rate constants k₁ and k₂ for the forward and reverse reactions of the initial step and k₃ and k₄ for the forward and reverse rate constants of the second step. Binding curves were calculated numerically and forward and reverse rate constants extracted by simulation at all analyte concentrations examined. Parallel model simulation of the forward reactions was done using summed exponentials; the observed pseudo first order rate constant k_{obs} is then the sum of the forward and reverse rates. In both cases the second order rate constant is the product of analyte concentration and pseudo first order forward rate constant k₁.

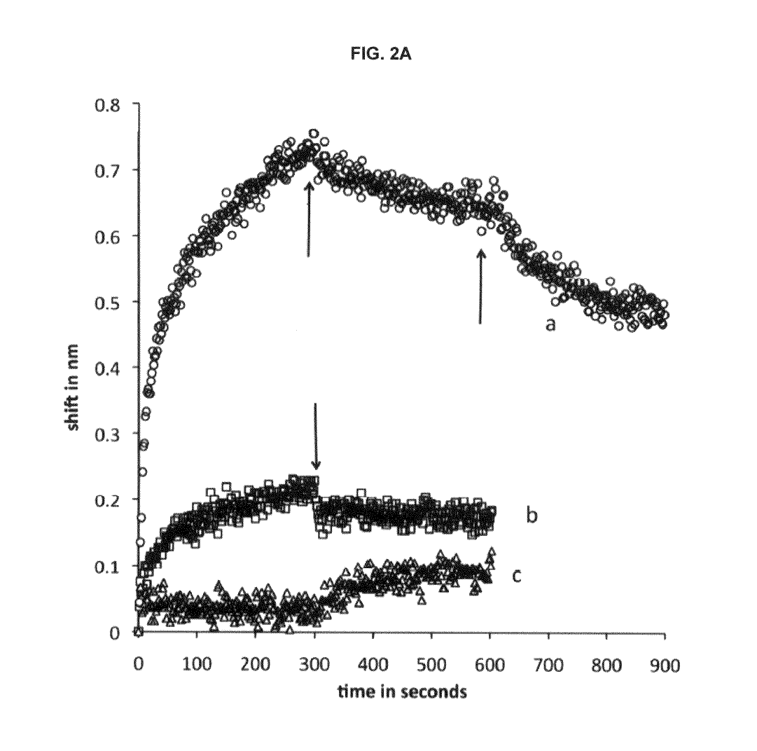

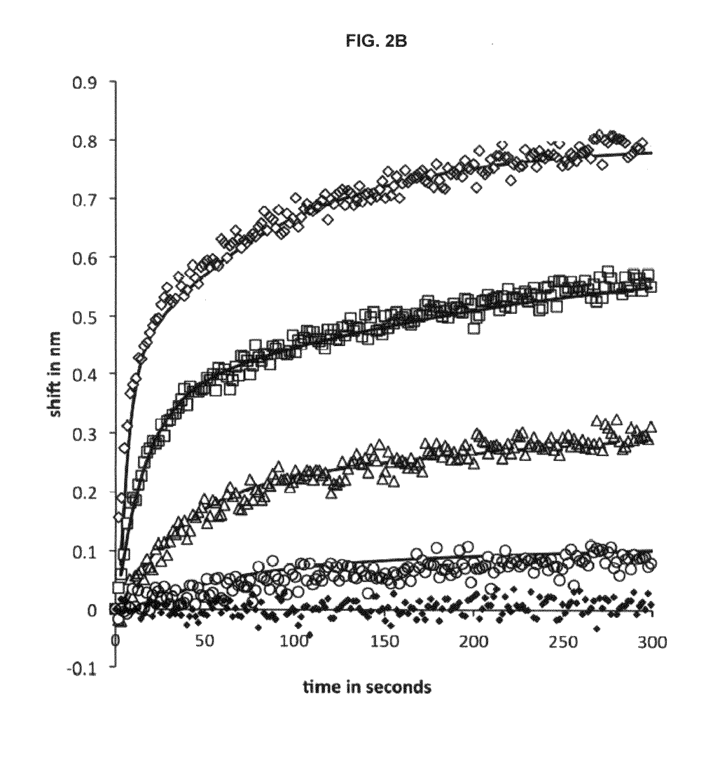

eNOS bound to p38. FIG. 2A trace a shows binding of eNOS to tethered p38, it shows sensorgrams of eNOS and nNOS binding to p38. Immobilized p38 was immersed in 696 nM NOS at time 0. Binding was measured for 300 s followed by transfer to buffer only and monitoring of dissociation for 300 s. Trace a (circles) was eNOS binding with an additional step in which the tip was moved into buffer with 1 μM CaM after initial dissociation. FIG. 2A trace b (squares) was nNOS binding. FIG. 2A trace c (triangles) was eNOS pre-equilibrated with a fourfold molar excess of CaM prior to immersion of p38. Arrows indicate movement of sensors from association to dissociation or dissociation to CaM-containing buffer.

Binding was rapid; an initial component accounts for about ⅔ of the signal and has a half time of about 10 sec. A second, slower component appears to represent changes in protein conformation on the sensor. After 300 s, the sensor was immersed in buffer only; dissociation of about 25% of the rapid phase binding was observed, consistent with conversion of the remaining complex to a slowly dissociating form in the second phase of association. After dissociation in buffer, the sensor was moved to buffer containing CaM. Dissociation was still incomplete, but significantly more rapid and extensive. The rapid phase of association was representative of potentially physiologically relevant events; very slow conformational changes of protein associated with the sensor surface likely represented partial unfolding or aggregation.

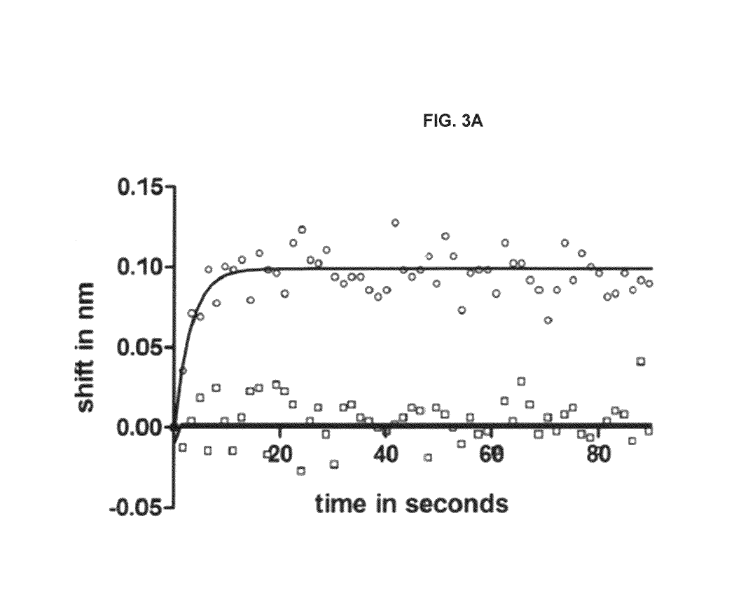

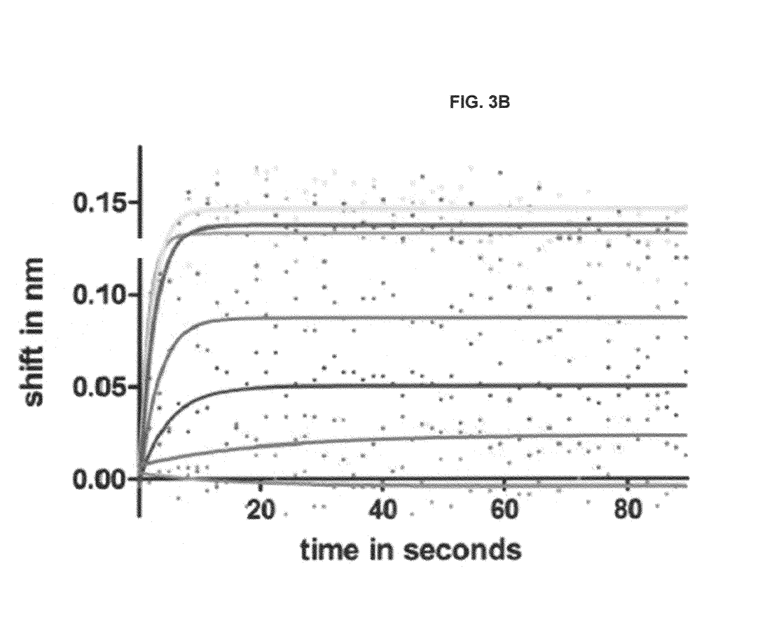

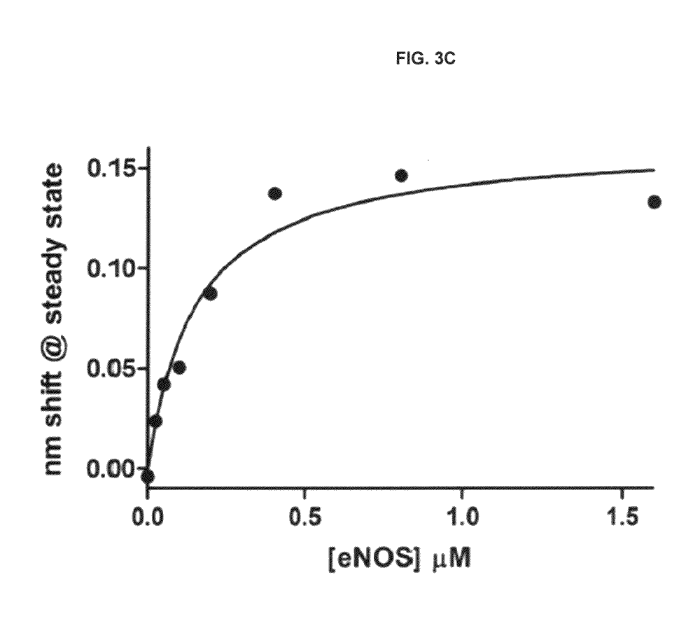

FIG. 2B shows sensorgrams of eNOS concentration course. eNOS concentrations in nM units were 696 (open diamonds), 232 (squares), 77 (triangles), 26 (circles) and 0 (black diamonds). Fits to a two-component sequential model are shown as solid lines. Dissociation phases were similar to trace a in FIG. 1. Kinetics parameter sets for successive traces in order of decreasing eNOS were 0.1, 0.01, 0..006, 0..001; 0.03, 0.01, 0.0002, 0.001; 0.0008. 0.01, 0.006, 0.001; 0.0025, 0.01, 0.0002, 0.001 in sec-1 for k1, k2. k3, and k4. B. Log k1 (rapid phase) vs. log [eNOS] demonstrated first order binding. Points were determined from simulations shown in FIG. 3A. Error bars represented the range of k1 that produced acceptable fits. Solid line was first order binding. Dotted line was second order binding.

FIG. 2B trace b shows the binding of nNOS to p38. The low amplitude of the signal compared to eNOS indicated that binding was an order of magnitude weaker, and binding was 30 fold slower. In FIG. 2B trace a eNOS was present at saturating concentrations, while the same concentration of nNOS was below the Kd; similar binding of eNOS can be obtained at concentrations about thirty fold lower (see titration in FIG. 2). Weak association of nNOS to p38 may be through basic residues in the Al or the CaM binding region, or may represent a different mode of interaction.

Mutants of eNOS have been previously studied in which the control elements have been deleted (28). Binding of an deletion mutant in which the autoinhibitory element has been removed to p38 is comparable to the binding of nNOS, but since the non-specific binding of the eNOS mutant is somewhat greater than that of wild type enzymes, it is not possible to draw more detailed conclusions about weak secondary binding sites. The results are consistent with the behavior of nNOS.

FIG. 2B trace c shows p38 immersed in eNOS as in trace a except that excess Ca⁺²/CaM was present during association. CaM abolishes binding, suggesting that binding sites overlap, consistent with recognition of the pentabasic motif. p38-eNOS interaction can also be observed with eNOS as ligand and p38 as free analyte (data not shown). The signal is much smaller; this was expected because eNOS is a large protein (dimer molecular weight about 260 kDa) and p38 is much smaller (molecular weight about 41 kDa). Inhibition by CaM was also observed. Reversibility of ligand and analyte demonstrated that the interaction was not an artifact of immobilization.

FIG. 2B shows concentration dependence of eNOS binding to p38. Simulation with two components produces good fits; a sequential fit is shown, but good fits can also be obtained with two parallel components. The rapid association phase, most likely to be physiologically relevant, was insensitive to choice of model. The magnitude and rate of eNOS binding that most closely matched the binding of nNOS in FIG. 1(b) was obtained at concentrations more than an order of magnitude lower.

Rapid binding is first order with respect to analyte (varied during titration), and the simulations are pseudo-first order with respect to immobilized ligand. Because the analyte was present in great excess, the observed binding kinetics are first order (in immobilized ligand) at each analyte concentration. At high eNOS concentrations the rate of binding (k₁) was much faster than the rate of release (k₂), hence the apparent rate constant k_{obs}˜k₁. The pseudo first order reaction rate at 232 nM eNOS was 0.03±0.003 sec⁻¹, corresponding to a diffusion limited rate constant of 0.13±0.01×10⁶ M⁻¹ sec⁻¹. K_{d} for eNOS binding to p38 was k₂/k₁, about 80±10 nM.

As shown in FIG. 2A, dissociation of eNOS from p38 was incomplete. This was not due to attainment of equilibrium in the rapid binding phase, because so little eNOS was bound to the sensor that full release would not produce a concentration in the dissociation buffer consistent with observable binding. These results were interpreted in terms of a sequential model in which the complex on the sensor was converted to a slowly released form. This process, occurring on the sensor over a time scale of several minutes, was unlikely to be biologically significant, but modeling it improved understanding of the fast phase.

eNOS bound to ERK. FIG. 3A shows the association phase of eNOS and nNOS binding to ERK-2. Immobilized ERK2 was immersed in 200 nM NOS at time 0. Trace a (circles) was eNOS binding. Trace b (squares) was nNOS binding. Solid lines indicate fits to a single exponential.

FIG. 3B shows sensorgrams of eNOS concentration course. eNOS concentrations in nM units were 1.6 μM (yellow), 0.8 μM (green), 0.4 μM (orange), 0.2 μM (purple), 0.1 μM (blue), 25 nM (red) and 0 (brown). Solid lines indicate fits to a single exponential.

FIG. 3C shows a plot of steady state amplitude against eNOS concentration. The fit shown is for K_{d}=160 nM.

FIG. 3A showed similar experiments demonstrating the binding of eNOS, but not nNOS, to ERK-2. The concentration dependence of eNOS binding to ERK-2 was shown in FIG. 3B. The concentration dependence was similar to that of the binding of eNOS to p38. The apparent K_{d} in the single component fit shown is about twice that measured for p38, but the data are consistent with K_{d} values as small as 90 nM (140±50 nM). A single kinetics component produces suitable fits for the initial 100 seconds of binding. Rapid binding is first order with respect to the analyte, and at each analyte concentration the observed kinetics are pseudo first order as in section 3.1. The pseudo first order reaction rate at 100 nM eNOS is 0.125±0.025 sec⁻¹, corresponding to a diffusion limited rate constant of 0.125±0.025×10⁶M⁻¹ sec⁻¹. Low amplitude, likely due to low binding activity of ligand, prevented accurate determination of off rates. Steady state analysis (FIG. 3C) of the binding in FIG. 3B yielded a K_{d} of 160 nM.

The effect of p38 binding on eNOS activity was investigated using the cytochrome c reduction assay for electron transfer and the hemoglobin capture assay for NO production. There was no significant effect on cytochrome c reduction. Weak inhibition of NO formation by 1 μM p38 in the presence of 0.6 μM CaM was observed; NO production was reduced by an average of 30%. This was consistent with competition between p38 and CaM, assuming that CaM is the stronger ligand as indicated in biosensor experiments. The protein-protein interaction alone does not appear to significantly affect eNOS activity.

The data demonstrated that p38 and ERK-2 bound eNOS with high affinity and bound nNOS only weakly or not at all. This identified the pentabasic sequence in the autoinhibitory element of eNOS, but not nNOS, as an important map kinase recognition site. Competition with CaM provided additional support for the pentabasic sequence as a MAP kinase target since these elements are proximally located in the three dimensional structure; displacement of the AI by CaM is the primary activating event in both eNOS and nNOS.

Physiological p38-eNOS binding has great potential in mediating signaling pathways known to involve both eNOS and p38. Other kinases might associate with the p38-eNOS complex rather than act as free intermediates. Trafficking of eNOS to cellular compartments is established (17-19), providing opportunity for differential interaction with p38 in some cell states. Evidence exists that MAP kinase pathways are involved in regulating both the expression and phosphorylation state of eNOS; some of these pathways inhibit and others activate NO synthesis (e.g., 30, 31) strongly suggesting a mixture of mechanisms that include pathway mediated effects and direct effects, e.g., direct phosphorylation of eNOS by ERK or p38, or activation of an intermediate activating kinase such as AKT in a multiprotein signaling complex.

Available evidence demonstrates that ERK directly phosphorylates eNOS in BAECs, and forms complexes with eNOS and additional components (11). These complexes are likely to be mediated at least in part by the interactions studied here. We point out that the location of the pentabasic motif, adjacent to the PKA phosphorylation site S633, suggests a mechanism of interaction between modes of inactivation and activation on the enzyme. Recently we directly confirmed inhibition of CaM binding after PKC phosphorylated T495, adjacent to the tribasic motif directly at the start of the CaM canonical target (21). It is of interest that the MAPK binding site is located in the Al adjacent to a comparable phosphorylation site; the MAPK site exposure is increased by Al displacement but MAPK competes with CaM, and the MAPK binding is positioned to interact with both PKA and AKT phosphorylation sites.

An alternative possibility with broader implications would involve additional partners. The pentabasic sequence of eNOS is recognized by a binding partner in mitochondria (32). Similar elements are involved in nuclear trafficking. It is easy to envision a system of alternating partners bearing base-rich sequences and their recognition sites that would enable kinases activated by MAP kinases to interact with partners and in turn, eNOS. Such interactions could create a phosphorylation cascade, but could also be the basis for inhibition. We are now investigating the potential of eNOS-MAPK interactions for enhancement of eNOS phosphorylation and inhibition of MAPKAP-2/3 kinases.

ERK directly phosphorylates eNOS in BAECs, and forms complexes with eNOS and additional components (11). These complexes are likely to be mediated at least in part by the interactions reported herein. The location of the pentabasic motif, adjacent to the PKA phosphorylation site S633, suggested a mechanism of interaction between modes of inactivation and activation on the enzyme. The inventor recently confirmed inhibition of CaM binding after PKC phosphorylated T495, adjacent to the tribasic motif directly at the start of the CaM canonical target (21). The MAPK binding site is located in the AI adjacent to a comparable phosphorylation site; the MAPK site exposure is increased by Al displacement but MAPK competes with CaM, and the MAPK binding is positioned to interact with both PKA and AKT phosphorylation sites.

The endothelial nitric oxide synthase bound MAP kinases at a pentabasic site in the unconserved region of the autoinhibitory insertion associated with the eNOS FMN binding domain. No potential MAPK phosphorylation sites are associated with this region in eNOS, but there are many good candidates elsewhere in the eNOS structure that may be accessible to a kinase bound at the pentabasic site. MAP kinases bind eNOS, but not homologs lacking the pentabasic site, with about 100 nM affinity and a forward rate constant of about 1.3×10⁵M⁻¹ sec⁻¹. Calmodulin forms a ternary complex that weakly promotes dissociation of p38 from eNOS. The eNOS-MAP kinase interaction may provide a scaffold for the formation of larger complexes with additional components, including Akt and Raf-1.

Endothelial nitric oxide synthase was inhibited by MAP kinase. Expression and purification of eNOS was carried out as previously described. Prosthetic group content was measured spectrophotometrically. NO synthase activity was measured using hemoglobin capture, and reductase activity was measured using cytochrome c reduction. ERK2 was purchased from SignalChem (Richmond, British Columbia Canada).

ERK kinase reactions were performed in 20 mM Hepes (pH 7.4), 1 mM DTT, 10 mM MgCl₂, 1 mM ATP, 10% glycerol, and when using the high concentration eNOS, 0.75 mM EGTA. eNOS was used at various concentrations (45 μM to 1.6 μM for fluorescence and eNOS/cytochrome C experiments, respectively). Reactions were done plus or minus ERK (0.12 μM to 0.01 μM, depending on eNOS concentration) at room temperature as described for the figures, and were ‘stopped’ by putting on ice until activity was tested, within 5 hours. 0.315 nMoles of eNOS phosphorylated and un-phosphorylated were analyzed by mass spectroscopy of trypsin digested enzyme (Emory University proteomics facility) with duplicate phosphorylated and unphosphorylated samples.

Fluorescence lifetime measurement was obtained with time-resolved intensity decays recorded using a PicoQuant Fluotime 100 time-correlated single-photon counting (TCSPC) fluorescence lifetime spectrometer as described (42). FMN was excited at 473 nm using a pulsed laser diode with 20 MHz repetition rate; experiments with 378 nm excitation produced similar results. The decay of fluorescence can be represented as the sum of individual exponential decays:

$$I ( t ) = {\sum }_{ii = 1}^{n} exp ( - t / {τ}_{i} ) \qquad ( 1 )$$

where the rᵢ are the decay times and αᵢ are the amplitudes of the i^{th} component. The fractional contribution of the i^{th} component in the steady-state is:

$${f}_{i} = \frac{{α}_{i} {τ}_{i}}{{\sum }_{j} {α}_{j} {τ}_{j}} \qquad ( 2 )$$

Individual values of αᵢ and rᵢ were determined from simulation with PTI's Felix GX software with PowerFit 10 simulation module, using deconvolution of an instrument response function obtained from scattering and nonlinear least squares fitting to multiple exponentials. The quality of the fits were characterized by χ² (48).

For optical biosensing, biolayer interferometry (BLI) experiments were conducted essentially as described. Briefly, biotinylated CaM was immobilized on streptavidin sensors. After establishing a baseline in binding buffer (10 mM HEPES, pH 7.4, 100 mM NaCl, 10% glycerol, 10 μM CaCl₂, 0.05% Surfactant P-20), sensors were moved to binding buffer containing eNOS for 180 s. Sensors were then moved to buffer only to monitor dissociation for 180 s. Nonspecific binding was measured by immersing sensors without CaM in analyte and was negligible in all cases.

Surface plasmon resonance (SPR) experiments were conducted on a Biacore X100 instrument using a biotin CAPture chip. Biotinylated CaM, about 150 RU in all cases, was immobilized prior to single-cycle kinetics performed in binding buffer.

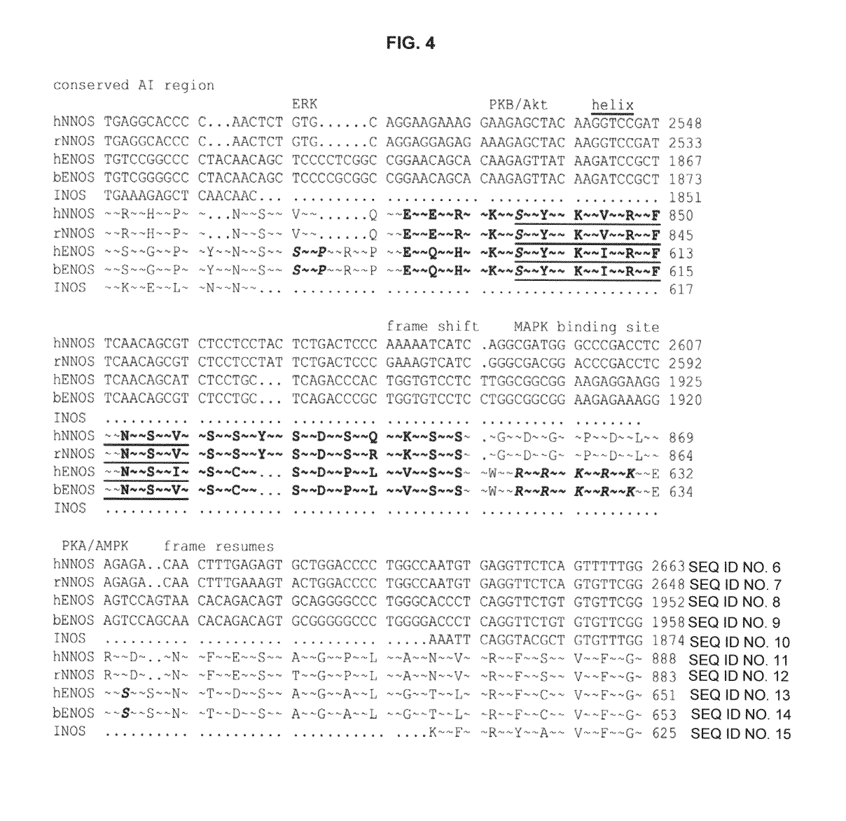

FIG. 4 shows the alignment of the DNA and amino acid sequences of mammalian nitric oxide synthase isoforms in the autoinhibitory element region, a region corresponding to an ab turn within the Rossman fold FMN binding domain. The conserved region (bold) and frame shifts caused divergence of the variable region in eNOS and nNOS. Helix residues are underlined. Phosphorylation sites for ERK, Akt, and PKA/AMPK, and the binding site for MAP kinases (ERK, P38) are marked with bold italics. Human sequences are denoted by h, rat sequences by n, and bovine sequences by b. All DNA sequences are numbered from the first base of the start codon in the most commonly studied splice form. DNA accession numbers: hnNOS, D16408; rnNOS, X59949; heNOS, M93718; beNOS, M99057; hiNOS, L24553. Protein accession numbers: hnNOS, P29475; rnNOS, P29476; heNOS, P29474; beNOS, P29473; hiNOS, P35228.

The signal generators eNOS and nNOS differ from the cytokine induced isoform iNOS in that they have an extended insertion at this point that serves as an autoinhibitory element (AI) displaced by calmodulin binding to a spatially adjacent site.

The N terminal half of the AI, shown in bold, contains a conserved helical region (underlined residues) that locks down the FMN binding domain through hydrogen bonds to both the FMN binding domain and the NADPH binding domain. This prevents conformational changes that are an obligatory part of the catalytic cycle. S617 and S635 in bovine eNOS (S615 and S633 in human eNOS) are phosphorylated by kinases (AKT and PKA) that activate the enzyme.

As described above, MAP kinases bind to a pentabasic sequence present in eNOS but not nNOS. This site, shown in bold italic, confers 50-100 picomolar affinity for p38 and ERK. The location in this regulatory region suggests interactions between MAP kinases and other modulators of eNOS activity.

The evolutionary origin of the MAP kinase binding site is implied by the frame shift in the eNOS sequence in comparison to nNOS at the end of the conserved AI region. A compensating shift restores the reading frame 27 bases later in eNOS, and homology at the amino acid level immediately resumes. During the 27 base pair frame shift, however, there is no relationship between eNOS and nNOS at the amino acid level; some homology appears to persist at the DNA level. Divergence of eNOS from nNOS occurred during or before the development of amphibians, and available amphibian sequences have either pentabasic or tetrabasic motifs. It is plausible that these motifs arose by chance and were stabilized during evolution by protein-protein interactions.

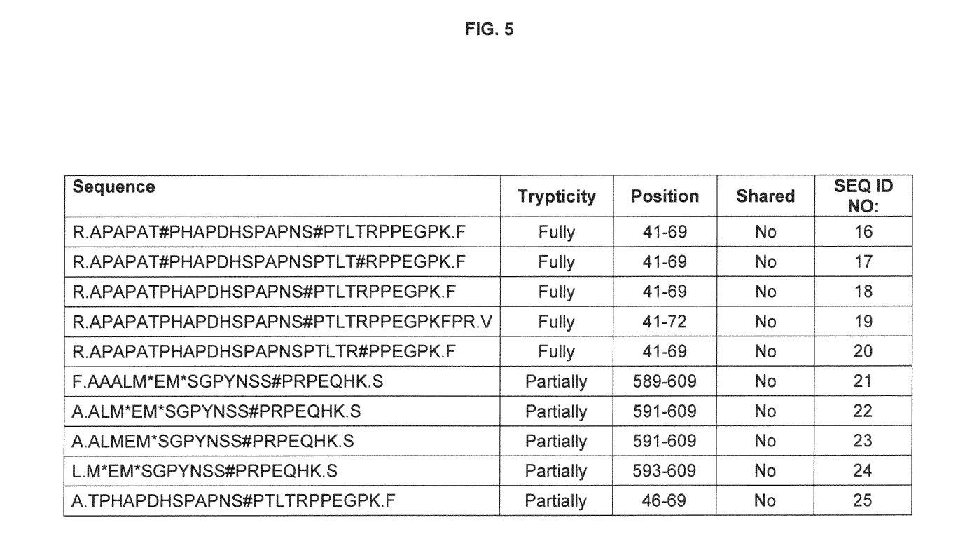

Phosphorylation of eNOS by ERK was detected in vitro by measurement of ADP. The results showed that over a one hour incubation two or three ADP were formed per eNOS monomer, suggesting that multiple sites were phosphorylated. FIG. 5 shows the results of mass spectroscopy of eNOS tryptic digests of unphosphorylated eNOS and eNOS phosphorylated for 15 minutes by ERK, showing phosphorylated peptides consistent with data. Phosphorylation sites are indicated by a hatch (#), and digestion locations are indicated by periods (.). Because ERK phosphorylates TP and SP motifs, the results are uniquely consistent with S602, T46, and S58 phosphorylation. Asterisks indicate oxidized methionines. No peptides were shared with the unphosphorylated control.

Coverage was essentially complete; 35 phosphorylated tryptic peptides were obtained in addition to 3545 unphosphorylated peptides.

S602 and T46 were identified unambiguously as phosphorylation sites from the mass spectroscopy results alone. The data showed that either S58 and T46 was phosphorylated; because MAP kinases phosphorylate at SP or TP sites, the third position was unambiguously identified as T62. No indication of phosphorylation on other sites was seen.

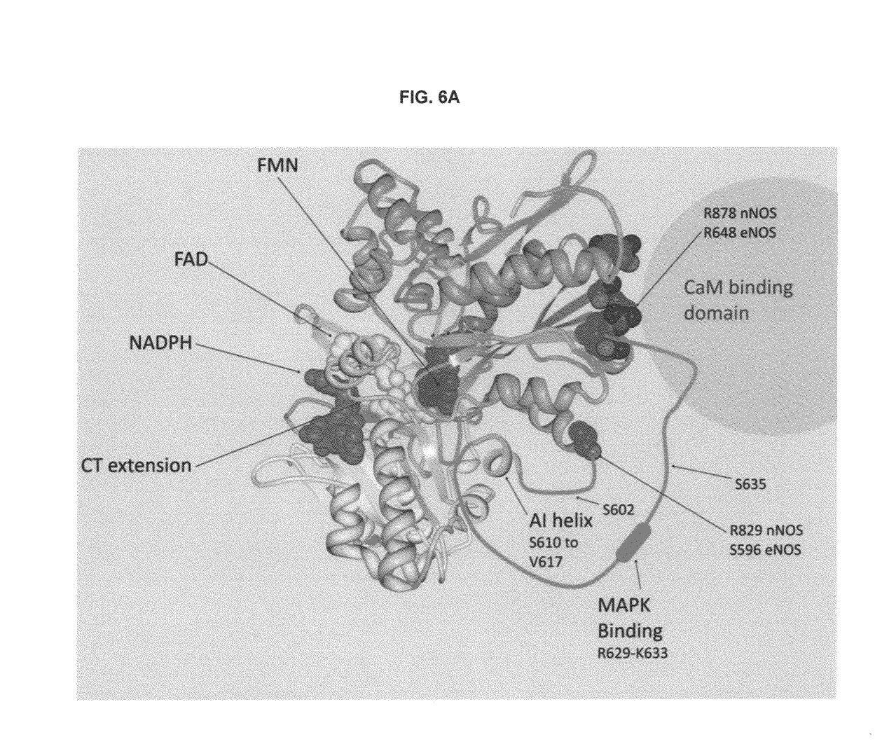

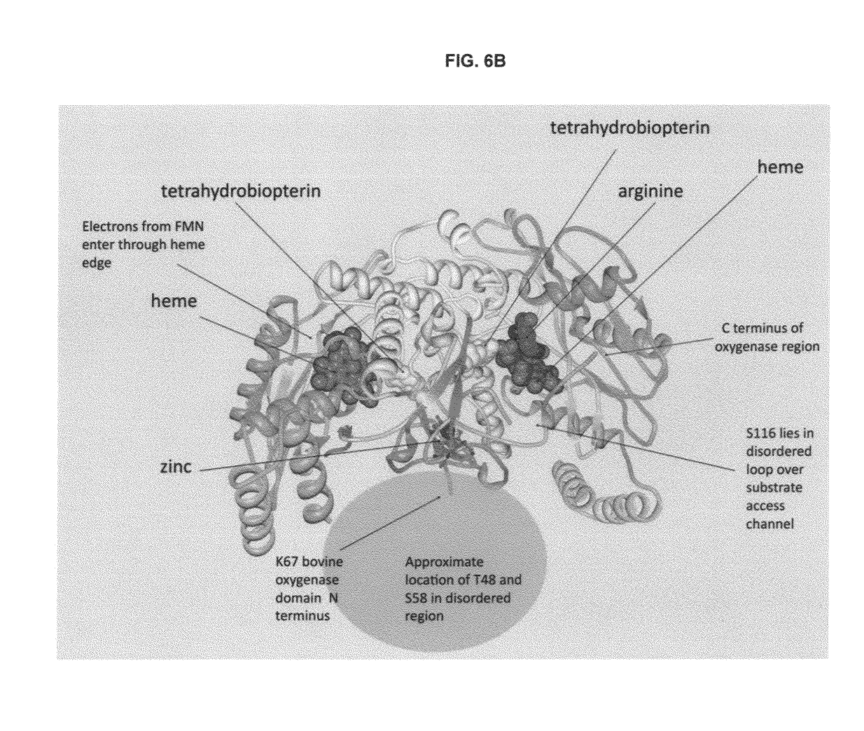

FIG. 6A is a structural cartoon of NOS reductase domains based on the crystal structure of nNOS reductase construct. The FMN binding domain is blue, the FAD binding domain green, and the NADPH binding domain is tan. Cofactors are shown in solid render, and the residues at the N terminal edge of the FMN binding domain and at the ends of the autoinhibitory insertion (R878/R648 and R829/S596 for nNOS/eNOS) are marked by solid render. The approximate extent and position of the disordered regions of the Al are indicated by a drawn coil. The autoinhibitory element of nitric oxide synthase (AI) carried previously identified targets for Akt and PKA phosphorylation as well as a MAP kinase binding site and a target for ERK phosphorylation (S602) that also matched the target motifs of other MAP kinases. The C terminal extension carries S1179, a target for Akt and PKA (see PDB 1TLL).

FIG. 6B is a structural cartoon of eNOS oxygenase dimer based on crystal structures. The direction of the backbone is indicated by color, with the N terminal blue and the ribbon shading through green to tan at the C terminal. Cofactors and substrate arginine are shown in solid render, with heme shown in red and tetrahydrobiopterin in yellow. The location of two ERK targets in a disordered region at the bottom of the figure is indicated; the region is near the dimer interface. The C terminal end of the domain is shown emerging from the face at right; this becomes the calmodulin binding site, and after a short connector joins the FMN binding domain shown in FIG. 3A. This domain must supply electrons to the heme at right by disengaging from the reductase complex and re-orienting (see PDB 4NSE and 3NOS). FIG. 6A shows the structure of the reductase portion of nNOS, corresponding roughly to the C terminal half of the enzyme. No corresponding eNOS crystal structure is available, but the two enzymes are highly homologous in this region and the conserved structures shown should be nearly identical.

The ribbon diagram traces the path of the backbone through the FMN, FAD, and NADPH binding domains, with the cofactors shown in solid render. In this conformation the FAD and FMN isoalloxazines are in Van der Waals contact. At the opposite edge of the FMN binding domain b sheet, the ends of the AI are exposed at the adjacent ends of an a helix and a b strand; the trace appears discontinuous because the chain is too flexible here to have a well-defined structure. The AI helix is visible as a disconnected feature, and is connected to the FMN domain beta sheet by a long disordered loop on the C terminal side and a short disordered loop on the N terminal side. The long loop carries the MAP kinase binding site close to the edge of the beta sheet and the adjacent CaM binding site, accounting for the CaM-MAP kinase competition we previously reported. S602 is located in the short disordered loop directly adjacent to the alpha helical region that precedes the AI. S617 is located in the Al helix, and S635 is in the long disordered loop between the MAP kinase binding site and the beta strand that follows the AI.

FIG. 6B shows the structure of the eNOS oxygenase dimer. The heme and tetrahydrobiopterin cofactors are shown in sold render along with the zinc atom that stabilizes the dimer. T46 and S58 are located in a disordered region adjacent to the loops bearing the cysteine residues that coordinate the zinc; the sites of myristoylation and palmitoylation are further towards the N terminal. This region of the oxygenase domain surface is close to the point where the polypeptide chain leaves the oxygenase domain to form the CaM binding site, and also reasonably close the site of heme reduction on the opposite monomer.

Table 1 summarizes experimental results showing the effect of ERK mediated phosphorylation on eNOS activity in NO synthesis and cytochrome c reduction. NO synthesis measured spectrophoto-metrically through the reaction of NO with oxyhemoglobin was inhibited by 50%. Reduction of ferricytochrome c by eNOS was monitored at 550 nm to assess the effect of phosphorylation on electron transfer within the reductase unit. Phosphorylation inhibited cytochrome c reduction by about 50%.

TABLE 1

| | NO | Cyto- | | | |
|---|---|---|---|---|---|
| | synthesis | chrome c | Fluorescence lifetime | | |
| | nm/min/mg | reduction | state populations | | |
| | enzyme | min¹ | 90 ps | 0.9 ns | 4.3 ns |
| Unphosphorylated | 110 (10) | 143 (20 | 53.45 | 20.21 | 26.35 |
| eNOS | | | | | |
| ERK phosphory- | 42 (7.6) | 55 (5) | 85 | — | 15 |
| lated eNOS | | | | | |

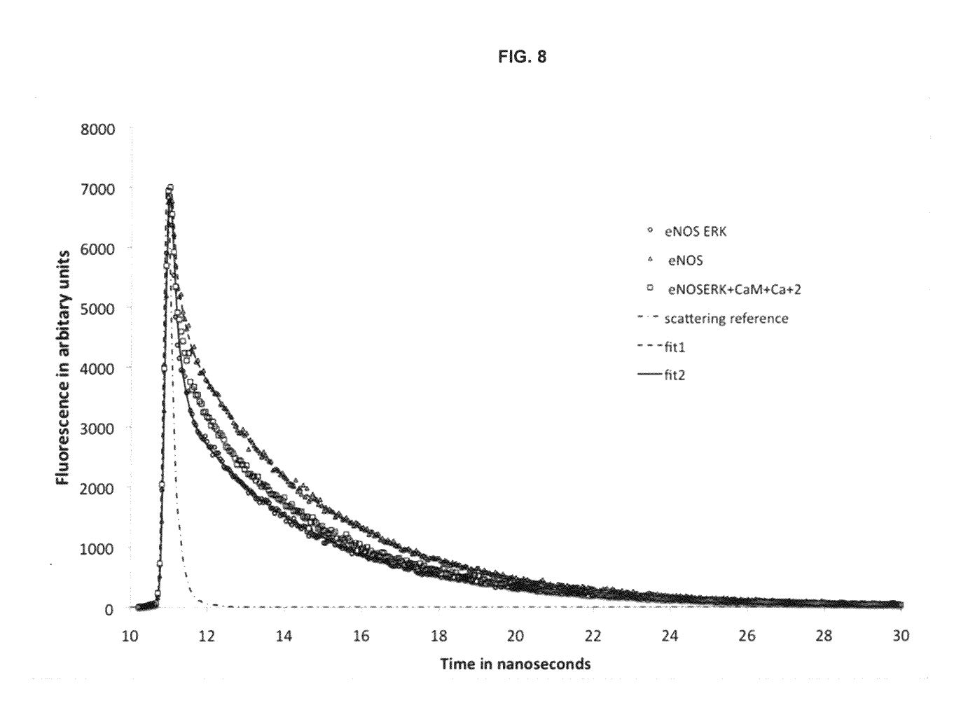

Effects of ERK catalyzed phosphorylation of eNOS on activity and on the population of eNOS conformational states characterized by FMN fluorescence lifetimes. The lifetime distributions in Table 1 differ somewhat from those in the fits shown in FIG. 8 because they are derived from decays of calmodulin activated enzyme to correspond to NO synthase activity. The distributions in FIG. 8 show the effects of phosphorylation in the absence of calmodulin.

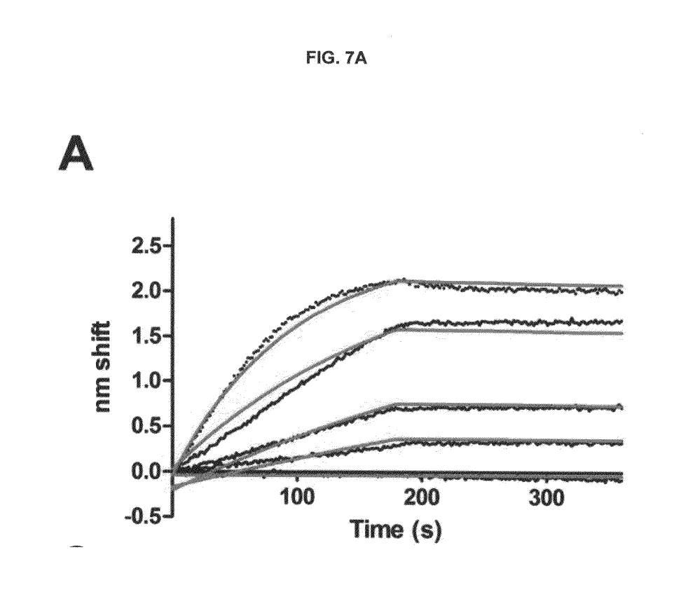

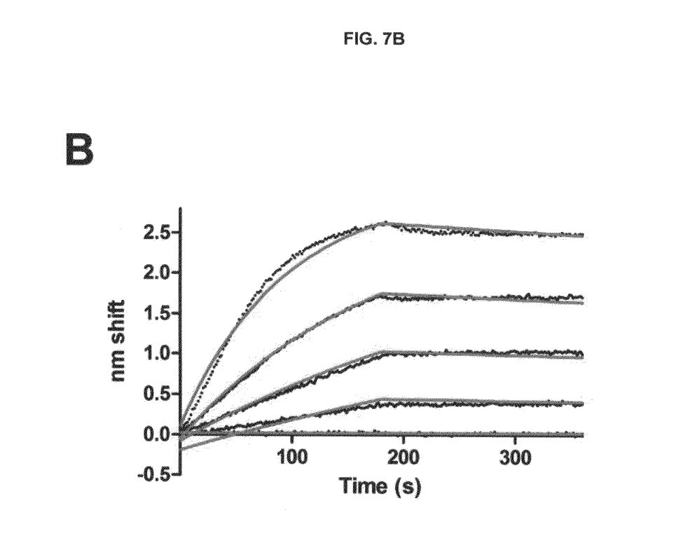

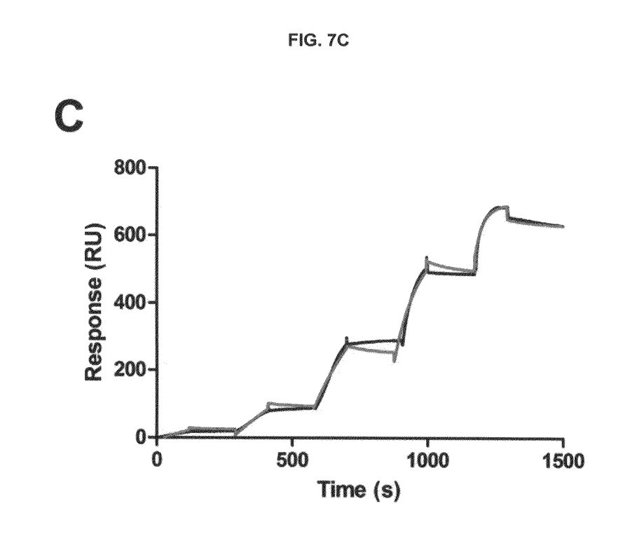

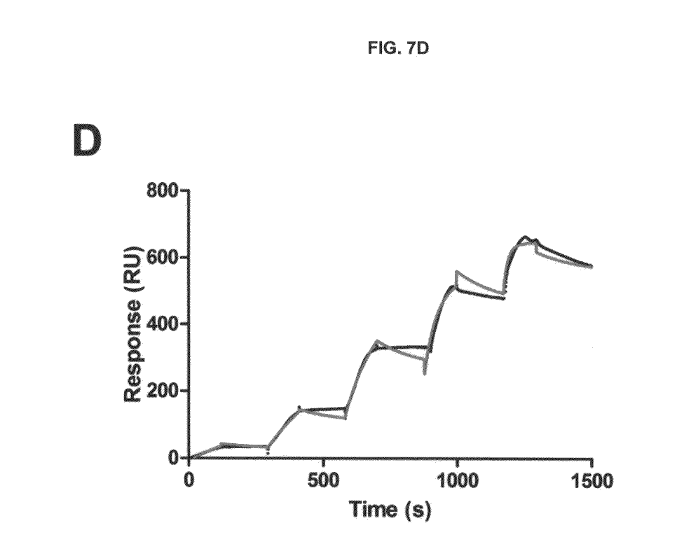

The inventor previously showed that CaM binding to unphosphorylated eNOS was diffusion limited and that PKC inhibition of eNOS via phosphorylation at T497 strongly inhibited CaM binding. ERK phosphorylation of the target residues does not strongly affect CaM binding, suggesting that PKC and ERK inhibit eNOS by different mechanisms. Optical biosensing experiments demonstrated near -1 nM affinities regardless of phosphorylation state. FIGS. 7A and 7B show BLI traces with fits to global single-state models for CaM binding to unphosphorylated and phosphorylated eNOS, respectively. Rate constants are shown in Table 2.

TABLE 2

| | Unphosphorylated | Phosphorylated |
|---|---|---|
| BLI (single state) | | |
| KₒₙM¹ S¹ | 1.2 × 10⁵ | 1.2 × 10⁵ |
| K_{off s}⁻¹ | 1.1 × 10⁻⁴ | 1.2 × 10⁻⁴ |
| K_{D}, pM | 920 | 2700 |
| SPR (Sequential model) | | |
| Kₒₙ₁M¹ S¹ | 6.4 ×. 10⁴ | 1.1 × 10⁵ |
| K_{off 1} S⁻¹ | 2.0 × 10⁻³ | 2.5 × 10⁻³ |
| K_{on 2} S⁻¹ | 8.4 × 10⁻³ | 3.7 × 10⁻³ |
| K_{off 2} S₁ | 1.9 × 10⁻⁴ | 1.1 × 10⁻⁴ |
| K_{D}, pM | 720 | 650 |

At the concentrations examined binding approximates single state with remarkably similar profiles regardless of phosphorylation. The difference in affinity observed was entirely due to a three-fold higher k_{off}, although this may not be physiologically relevant.

FIG. 7 shows an optical biosensing analysis of CaM-eNOS binding. FIG. 7A is a BLI sensorgram of CaM binding to 0 nM, 10.9 nM, 31.9 nM, 43.7 nM, and 87.5 nM unphosphorylated eNOS. Raw data are in black. Fits to a single-state association-then-dissociation model are shown in red. FIG. 7B show the same as in FIG. 7A, but for the same concentrations of phosphorylated eNOS. FIG. 7C is a SPR sensorgram of single-cycle kinetics of tethered CaM binding to 6.2 nM, 18.5 nM, 55.6 nM, 167 nM, and 500 nM analyte unphosphorylated eNOS. Reference-subtracted raw data are in shown in black along with fits to a two-state sequential model in red. FIG. 7D show the same as in FIG. 7C, but with phosphorylated eNOS.

Single-cycle kinetic analysis by SPR also revealed that CaM binding of unphosphorylated eNOS (FIG. 7C) was highly similar to phosphorylated (FIG. 7D). CaM was immobilized prior to injection of analyte eNOS. Fits to a sequential model (A+B⇄AB⇄AB*) generated K_{D}s similar to those determined from BLI (Table 1), indicating that the secondary event was a minor component.

The inventor recently showed that iNOS passes through a series of obligatory conformational states during its catalytic cycle, including an input state in which the FMN binding domain is closely associated with the FAD and NADPH binding domains, an output state in which the FMN binding domain is associated with the heme containing oxygenase domain, and a series of open conformations in which FMN is not closely coupled to other prosthetic groups. These states can be resolved by their very different fluorescence lifetimes; eNOS and nNOS have similar conformational states. Calmodulin activation of eNOS and nNOS resulted in increased levels of the output and open states relative to the closed input state.

FIG. 8 shows fluorescence decays of unphosphorylated and ERK phosphorylated eNOS holoenzyme FMN excited at 473 nm and detected at 525 nm in the presence and absence of calmodulin. The eNOS concentrations in the samples were 1 μM, and were from the same preparation. ERK phosphorylation favors conformational states with shorter lifetimes; calmodulin activation produces the opposite effect (not shown). Calmodulin partially reverses the effect of ERK phosphorylation, but the effect of calmodulin binding to ERK phosphorylated NOS was much smaller than the effect of calmodulin on the unphosphorylated enzyme. Fitting parameters were as follows. For unphosphorylated eNOS 90 ps, 78%; 3.65 ns, 20%; 0.9 ns, 0.68%, 10.1 ns, 1.6%; c²=1.10. For ERK phosphorylated eNOS: 80 ps, 86%, 3.83 ns, 12.4%, 1.1 ns 1.8%. All experiments were performed six times using at least two different preparations. The scattering reference was collected at 475 nm, and indicated the width of the exciting pulse

ERK phosphorylation caused an increase in the input state, which has a lifetime of 90 ps because of close coupling between FMN and FAD, and a concomitant increase in the output state (0.9 ns lifetime) and the open states (4.3 ns average lifetime). This effect is in opposition to the effect of calmodulin binding, and accounts for the inhibition of NO synthesis and cytochrome c reduction by ERK mediated phosphorylation.

The effects of ERK phosphorylation on the activity of eNOS, and on the distribution of conformational states characterized by their FMN fluorescence lifetimes, is summarized in Table 1. The effects of ERK phosphorylation on the conformational manifold are opposite those of the principal activator, calmodulin. Inhibition of both NO synthesis and cytochrome c reduction was at least 60%, and may be much greater because eNOS could not be completely phosphorylated without long incubations that damage the enzyme.

The MAP kinases ERK and P38 are believed to participate in negative feedback signaling networks with eNOS, and good evidence has been obtained for direct phosphorylation of eNOS by ERK in BAECs. Based on indirect evidence obtained in intact cells, several sites have been proposed for ERK phosphorylation including S116, T497, and S635. Of these sites, only S116 has the SP/TP motif generally associated with MAP kinase targets. S116 is located on the oxygenase domain, and the MAP kinase binding site is located on the autoinhibitory element associated with the FMN binding domain.

Direct in vitro phosphorylation of purified eNOS with purified ERK confirmed that eNOS is a substrate for ERK, and further showed that phosphorylation did not require additional scaffolds or adapters. ERK phosphorylation inhibits both NO synthesis and cytochrome c reduction. This indicated that phosphorylation interfered with electron transfer reactions mediated by FMN.

Three sites of phosphorylation on eNOS were identified. S602, at the N terminal end of the AI, is spatially adjacent to both the MAP kinase binding site and the CaM binding site, and is well positioned to interact with other control sites. A ‘lockdown’ of the FMN binding domain was suggested by fluorescence results showing that phosphorylation pulls the enzyme's conformational distribution toward the input state. This accounts for the observed inhibition, and suggests that the negatively charged phosphate group interacts with positively charged residues to stabilize the input conformation. CaM appears to bind strongly to ERK phosphorylated eNOS, but was unable to effectively override phosphorylation imposed inhibition. Although the results of CaM activation and ERK phosphorylation on the fluorescence profile are fortuitously opposite, effects on the conformational equilibria are secondary to the changes in rates for conformational transitions. It is possible to inhibit the enzyme by locking it into any conformation, because the mechanism depends on conformational cycling.

The complex kinetics of CaM-eNOS binding are disclosed. ERK-phosphorylated eNOS demonstrated a highly similar CaM-binding profile: diffusion-limited association, very slow dissociation, and picomolar affinity. A threefold slower dissociation rate constant in BLI accounted for all of the affinity difference between unphosphorylated and phosphorylated eNOS. The difference may be kinetically significant (95% confidence intervals for k_{off} do not overlap) but cannot account for differences in regulation of NOS activities. Other factors, e.g. aging of complexes on the BLI sensors, may vary with phosphorylation and account for the observed differences. Indeed, the difference in k_{off} was much less pronounced in SPR and the affinities were nearly identical to the 650 μM.

ERK and p38 phosphorylation of eNOS had no measurable effect on the binding of eNOS to calmodulin. In contrast, phosphorylation of T497 by PKC interfered with CaM binding. The modes of ERK and PKC inhibition are thus entirely different: PKC prevented the binding of an activator, while ERK interrupted the catalytic cycle.

ERK also phosphorylates eNOS at T46 and S58; these residues are located in a disordered region on the surface of the oxygenase domain. Because ERK phosphorylates all three residues, their effects cannot yet be separated. The location of S602 is ideal for the control of FMN mediated electron transfer, and it lies close to the D domain type MAP kinase binding site. The mode of action of T46 and S58 phosphorylation is unclear; because the disordered N terminal region is known to participate in protein-protein interactions and protein trafficking, these sites may be signals that affect protein complex formation, myristolation/palmitoylation, and targeting to specific cellular compartments rather than direct regulators of activity, but they could also affect oxygenase domain function. It is unclear that ERK bound to the Al pentabasic site can phosphorylate sites on the oxygenase domain. Given that both the AI loop that carries this site and the target region are flexible, this may not be impossible, but it is possible that a secondary biding site exists that facilitates phosphorylation at T46 and S58.

It is unlikely that MAP kinases phosphorylate residues such as T497, S617,S635, and S1179. All these sites, with the exception of T497, activate eNOS, and none of them has an SP or TP target motif. When phosphorylation of these sites correlates with MAP kinase activation, it is likely that this occurs in a pathway dependent manner. For inhibitory events such as T497 phosphorylation, this might represent parallel pathways of inhibition. For activating events, it probably represents feedback pathways involved in push-pull regulation.

S116 was not detected in phosphorylation. This residue is located on a flexible loop near the mouth of the substrate access channel on the surface of the oxygenase domain, and is known to be phosphorylated by SP directed kinases in vivo. Without being bound by a single theory, it is possible that phosphorylation could be mediated by other kinases, or phosphorylation by MAP kinases could require additional input (e.g., prior phosphorylation of other sites), or phosphorylation by MAP kinases could require scaffolding components.

The tight spacing of control sites on the eNOS surface indicated a system in which eNOS is the junction of many signaling pathways and an active node in the resulting network. A simple example of multiple inputs is T497 phosphorylation, which interferes with CaM binding and hence activation. The sites associated with the Al are far more complex. Activating kinases such as PKA and AKT phosphorylate multiple targets in this region, which also includes the ERK/p38 binding site, the S602 ERK target, and the autoinhibitory helix, which locks the FMN binding domain to the NADPH binding domain. It is likely that phosphorylation of these sites affects binding of other regulators and phosphorylation of other sites. The complexity of the system may allow eNOS to behave more like an integrated circuit with multiple inputs and a nuanced array of outputs than a simple relay.

Applicants incorporate by reference the material contained in the accompanying computer readable Sequence Listing identified as Sequence_Listing_ST25.txt, having a file creation date of Feb. 15, 2013, 11:33 a.m., and file size of 10.1 kilobytes.

Each of the following are expressly incorporated by reference herein in its entirety:
1. Cargnello, M., and Roux, P. P. (2011) Activation and function of the MAPKs and their substrates, the MAPK-activated protein kinases, Microbiol. Mol. Biol. Rev. 75, 50-83.
2. Cuadrado, A., and Nebreda, A. R. (2010) Mechanisms and functions of p38 MAPK signaling, Biochem. J. 429, 403-417.
3. Zarubin, T., and Han, J. (2005) Activation and signaling of the p38 MAP kinase pathway, Cell Res. 15, 11-18.
4. Bardwell, A. J., Frankson, E., and Bardwell, L. (2009) Selectivity of docking sites in MAPK kinases, J. Biol. Chem. 284, 13165-13173.
5. Gaestel, M. (2008) Specificity of signaling from MAPKs to MAPKAPKs: kinases' tango nuevo, Front. Biosci. 13, 6050-6059.
6. Ignarro, L. J., Buga, G. M., Wood, K. S., Byrns, R. E., and Chaudhuri, G. (1987) Endothelium-derived relaxing factor produced and released from artery and vein is nitric oxide, Proc. Natl. Acad. Sci. 84, 9265-9269.
7. Furchgott, R. (1988) Studies in relaxation of rabbit aorta by sodium nitrite: the basis for the proposal that the acid activatible factor from bovine retractor penis is inorganic nitrite, in Vasodilation: Vascular Smooth Muscle Peptides, Autonomic Nerves and Endothelium, pp 401-404, Raven Press, New York.
8. Papapetropoulos, A., Garcia-Cardena, G., Madri, J. A., and Sessa, W. C. (1997) Nitric oxide production contributes to the angiogenic properties of vascular endothelial growth factor in human endothelial cells, J. Clin. Invest. 100, 3131-3139.
9. Corbett, J. A., Sweetland, M. A., Wang, J. L., Lancaster, J. R., Jr., and McDaniel, M. L. (1993) Nitric oxide mediates cytokine-induced inhibition of insulin secretion by human islets of Langerhans, Proc. Natl. Acad. Sci. 90, 1731-1735.
10. Henningsson, R., Salehi, A., and Lundquist, I. (2002) Role of nitric oxide synthase isoforms in glucose-stimulated insulin release, Am. J. Physiol. 283, C296-304.
11. Bernier, S. G., Haldar, S., and Michel, T. (2000) Bradykinin-regulated Interactions of the Mitogen-activated Protein Kinase Pathway with the Endothelial Nitric-oxide Synthase, J Biol. Chem. 275, 30707-30715
12. Roux, P. R., and Blenis, J. (2004) ERK and p38 MAPK-Activated Protein Kinases: a Family of Protein Kinases with Diverse Biological Functions, Microbiol. Mol. Biol. Rev. 68, 320-344
13. Enslena, H., Davis, H.J. Regulation of MAP kinases by docking domains, Biology of the Cell 93 (2001) 5-14.
14. Sharrocks, A. D. Yang, S.-I. and Galanis, A. (2000) Docking domains and substrate specificity determination for MAP kinases, TIBS 25, 448-453.
15. Bardwell, A. J., Frankson, E. and Bardwell, L. (2009) Selectivity of Docking Sites in MAPK Kinases J. Biol. Chem. 284, 13165-13173
16. Salerno, J. C., Harris, D. E., Irizarry, K., Patel, B., Morales, A. J., Smith, S. M., Martasek, Roman, L. J., Masters, B. S., Jones, C. L., Weissman, B. A., Lane, P., Liu, Q., and Gross, S. S. (1997) An autoinhibitory control element defines calcium-regulated isoforms of nitric oxide synthase, J. Biol. Chem. 272, 29769-29777.
17. Boo, Y. C., Kim, H. J., Song, H., Fulton, D., Sessa, W., and Jo, H. (2006) Coordinated regulation of endothelial nitric oxide synthase activity by phosphorylation and subcellular localization, Free Rad. Biol. & Med. 41, 144-153.
18. Mount, P. F., Kemp, B. E., and Power, D. A. (2007) Regulation of endothelial and myocardial NO synthesis by multi-site eNOS phosphorylation, J. Mol. Cell. Cardiology 42, 271-279.
19. Fulton, D., Gratton, J. P., and Sessa, W. C. (2001) Post-translational control of endothelial nitric oxide synthase: why isn't calcium/calmodulin enough?, J. Pharmacology And Experimental Therapeutics 299, 818-824.
20. Garcin, E. D., Bruns, C. M., Lloyd, S. J., Hosfield, D. J., Tiso, M., Gachhui, R., Stuehr, D. J., Tainer, J. A., and Getzoff, E. D. (2004) Structural basis for isozyme-specific regulation of electron transfer in nitric-oxide synthase, J. Biol. Chem. 279, 37918-37927.
21) McMurry J L, Chrestensen C A, Scott I M, Lee E W, Rahn A M, Johansen A M, Forsberg B J, Harris K D, Salerno J C. (2011) Rate, affinity and calcium dependence of nitric oxide synthase isoform binding to the primary physiological regulator calmodulin. FEBS J. 278, 4943-54.
22. Gerber, N. C., and Ortiz de Montellano, P. R. (1995) Neuronal nitric oxide synthase. Expression in Escherichia coli, irreversible inhibition by phenyldiazene, and active site topology, J. Biol. Chem. 270, 17791-17796.
23. Roman, L. J., Sheta, E. A., Martasek, P., Gross, S. S., Liu, Q., and Masters, B. S. (1995) High-level expression of functional rat neuronal nitric oxide synthase in Escherichia coli, Proc. Natl. Acad. Sci. 92, 8428-8432.
24. Martasek, P., Liu, Q., Liu, J., Roman, L. J., Gross, S. S., Sessa, W. C., and Masters, B. S. (1996) Characterization of bovine endothelial nitric oxide synthase expressed in E. coli, Biochem. Biophys. Res. Com. 219, 359-365.
25. Gross, S. S. (1996) Microtiter plate assay for determining kinetics of nitric oxide synthesis, Methods Enz. 268, 159-168.
26. Newman, E., Spratt, D. E., Mosher, J., Cheyne, B., Montgomery, H. J., Wilson, D. L., Weinberg, J. B., Smith, S. M., Salerno, J. C., Ghosh, D. K., and Guillemette, J. G. (2004) Differential activation of nitric-oxide synthase isozymes by calmodulin-troponin C chimeras, J. Biol. Chem. 279, 33547-33557.
26. Chrestensen, C. A., Schroeder, M. J., Shabanowitz, J., Hunt, D. F., Pelo, J. W., Worthington, M. T., and Sturgill, T. W. (2004) MAPKAP kinase 2 phosphorylates tristetraprolin on in vivo sites including Ser178, a site required for 14-3-3 binding, J. Biol. Chem. 279, 10176-10184.
27. Abdiche, Y., Malashock, D., Pinkerton, A., and Pons, J. (2008) Determining kinetics and affinities of protein interactions using a parallel real-time label-free biosensor, the Octet, Anal. Biochem. 377, 209-217.
28. Roman, L. J., Masters, B. S. (2006) Electron transfer by neuronal nitric-oxide synthase is regulated by concerted interaction of calmodulin and two intrinsic regulatory elements J. Biol. Chem. 281, 23111-23118

029. Lukas, S. M., Kroe, R. R., Wildeson, J., Peet, G. W., Frego, L., Davidson, W., Ingraham, R. H., Pargellis, C. A., Labadia, M. E., and Werneburg, B. G. (2004) Catalysis and function of the p38 alpha MK2a signaling complex, Biochem. 43, 9950-9960.
30. Kumar, V. B., Viji, R. I., Kiran, M. S., Sudhakaran ,P.R. (2009) Negative modulation of eNOS by laminin involving post-translational phosphorylation. J Cell Physiol. 219(1):123-31.
31. Kan W H, Hsu J T, Ba Z F, Schwacha M G, Chen J, Choudhry M A, Bland K I, Chaudry I H. (2008) p38 MAPK-dependent eNOS upregulation is critical for 17beta-estradiol-mediated cardioprotection following trauma-hemorrhage. Am J Physiol Heart Circ Physiol. 294, H2627-36.
32. Gao, S., Chen, J., Brodsky, S. V., Huang, H., Adler, S., Lee, J. H., Dhadwal, N., Cohen-Gould, L., Gross, S. S., and Goligorsky, M. S. (2004) Docking of endothelial nitric oxide synthase (eNOS) to the mitochondrial outer membrane: a pentabasic amino acid sequence in the autoinhibitory domain of eNOS targets a proteinase K-cleavable peptide on the cytoplasmic face of mitochondria, J. Biol. Chem. 279, 15968-15974.

What is claimed is:

1. A method for modulating nitric oxide synthase activity, the method comprising contacting a nitric oxide synthase (NOS) with an effective amount of a mitogen-activated protein (MAP) kinase, wherein contacting the NOS with the MAP kinase results in phosphorylation of at least one amino acid selected from the group of S602, T46, and S58 resulting in NOS activity modulation.

2. The method of claim 1 wherein the MAP kinase is selected from the group consisting of extracellular signal-regulated kinase (ERK), p38 MAP kinase, and c-Jun N-terminal kinase (JNK).

3. The method of claim 2 wherein the MAP kinase is ERK.

4. A method for modulating the cytochrome C reductase activity of a nitric oxide synthase (NOS), the method comprising contacting a NOS reductase domain with an effective amount of a mitogen-activated protein (MAP) kinase to result in phosphorylation of at least one amino acid from the NOS reductase results in cytochrome C reductase activity modulation.

5. The method of claim 4 wherein the MAP kinase is selected from the group consisting of extracellular signal-regulated kinase (ERK), p38 MAP kinase, and c-Jun N-terminal kinase (JNK).

6. The method of claim 4 wherein the MAP kinase is ERK.

7. A polypeptide comprising a pentabasic site, wherein the pentabasic site is in an autoinhibitory element in an endothelial nitric oxide synthase (eNOS) and the pentabasic site is capable of binding a peptide that is a mitogen activated protein (MAP) kinase.

8. The polypeptide of claim 7 wherein the peptide capable of being bound inhibits binding of a MAP kinase.

9. The polypeptide of claim 7, wherein the polypeptide is endothelial nitric oxide synthase.

10. The polypeptide of claim 9, wherein the pentabasic site is in the un-conserved region of the autoinhibitory insertion associated with the eNOS FMN binding domain.

11. A peptide capable of binding to the pentabasic site of the polypeptide of claim 7.

12. An antibody capable of binding to the pentabasic site of the polypeptide of claim 7.

13. The method of claim 7 wherein the pentabasic binding site has a function selected from the group consisting of a target of phosphorylation, a modulator of NOS activity, and combinations thereof.

14. A method for modulating nitric oxide synthase (NOS) activity, the method comprising
contacting a nitric oxide synthase with an effective amount of peptide for a time sufficient to allow binding of the nitric oxide synthase with the peptide;
wherein contacting the NOS with the peptide results in the binding of the peptide to a pentabasic sequence in an auto-inhibitory element of the NOS, and wherein binding of the peptide to the NOS results in NOS activity modulation.

15. The method of claim 14 wherein the peptide is a mitogen activated protein (MAP) kinase.

16. The method of claim 14 wherein contacting the NOS synthase with the MAP kinase results in phosphorylation of at least one amino acid selected from the group consisting of S602, T46, and S58, and wherein the phosphorylation results in NOS activity modulation.

17. The method of claim 14 wherein NOS activity is increased.

18. The method of claim 11, wherein NOS activity is decreased.

19. A method for modulating nitric oxide synthase activity, the method comprising
contacting a nitric oxide synthase (NOS) with an effective amount of a mitogen activated protein (MAP) kinase for a time sufficient to allow binding of the NOS with the MAP kinase;
wherein contacting the nitric oxide synthase with the MAP kinase results in binding the MAP kinase to a pentabasic sequence in an auto-inhibitory element of the NOS; and
wherein binding of the MAP kinase to the NOS results in NOS activity modulation.

20. The method of claim 19 wherein contacting the NOS with the MAP kinase results in phosphorylation of at least one amino acid selected from the group consisting of S602, T46, and S58, wherein the phosphorylation results in NOS activity modulation.

21. A method for regulating angiogenesis and/or vascular tone, the method comprising providing a mitogen-activated protein (MAP) kinase to a biological system under conditions sufficient to regulate nitric oxide synthase (NOS) by binding the MAP kinase to a pentabasic site in a endothelial NOS autoinhibitory element, thereby resulting in for regulation of angiogenesis and/or vascular tone.

22. The method of claim 21 wherein the MAP kinase is selected from the group consisting of Extracellular signal-regulated kinase (ERK), p38 MAP Kinase and c-Jun N-terminal kinase (JNK).
